(19) United States
(12) Patent Application Publication
(10) Pub. No.: US 2007/0005048 A1
(43) Pub. Date: Jan. 4, 2007

(54) Method and apparatus for cryogenically treating lesions on biological tissue

(76) Inventors: R. Sam Niedbala, Allentown, PA (US); Alexander Volkov, Allentown, PA (US)

Correspondence Address: NIXON PEABODY, LLP, 401 9TH STREET, NW, SUITE 900, WASHINGTON, DC, 20004-2128, US

(21) Appl. No.: 11/476,901

(22) Filed: Jun. 29, 2006

Related U.S. Application Data
(60) Provisional application No. 60/694,929, filed on Jun. 30, 2005.

(51) Int. Cl. A61B 18/04

(52) U.S. Cl. 606/22

(57) ABSTRACT

The invention relates to a method, a cryosurgical probe, and a cryosurgical instrument for freezing a lesion on biological tissue. The method involves repeated cycles of freezing and thawing of the tissue to ensure maximum treatment effectiveness. The cryosurgical probe may be positioned on an end of a cryosurgical instrument, and facilitates the freezing of a lesion on biological tissue via the evaporation of a refrigerant. The cryosurgical probe includes a receiving end, a hollow portion, and a tissue contact end. The receiving end is adapted to receive the refrigerant. The tissue contact end includes an orifice adapted to expose the lesion to the refrigerant, and preferably includes a guard portion adapted to prevent excessive exposure of the biological tissue surrounding the lesion to the refrigerant. The hollow portion allows the flow of refrigerant from the receiving end to the tissue contact end, and includes at least one exhaust port.

### RELATED APPLICATION DATA

This application claims the benefit of Provisional Application Ser. No. 60/694,929, filed Jun. 30, 2005, the entire disclosure of which is incorporated herein by reference.

### FIELD OF THE INVENTION

The invention relates to the cryogenic freezing of lesions on biological tissue, more specifically, to a method and apparatus for cryogenically treating topical warts and lesions on humans and animals.

### BACKGROUND

The treatment of common warts and superficial lesions on human and animal skin is accomplished through a number of methods. These may include cuterage, acid treatments and cryo surgery.

Among the problems associated with scalpel surgery (i.e. cuterage) are (1) bacterial skin infection rates of up to about 18% depending upon, among other things, the sterile technique employed by the operator and heat and humidity of the location of the surgery, (2) hypertrophic scarring which can occur in up to about 25% of patients depending, in general, upon the operator's skill, experience and judgement, and the patient's genetic predisposition to scar, and (3) inefficient use of time. Most scalpel surgery procedures generally require about 25 to 50 minutes to perform. This time is necessitated by the time required for (a) anaesthetizing the treatment area, (b) about 5 minutes waiting period for the anesthetic, e.g. lidocaine, to become optimally effective, (c) time for preparing a sterile operating field, and (d) time for performing the scalpel surgery procedure.

Among the problems associated with electrodesiccation are (1) time consuming need for a local anesthetic to be applied and become optimally effective and (2) permanent hypertrophic scarring that occurs in a significant percentage of patients undergoing this procedure.

An example of the literature discussing some of these prior art methods and corresponding problems is Skin Surgery, Irwin Epstein and Irwin Epstein, Jr., 6th edition, 1987, W. B. Saunders, Philadelphia, Pa., pages 180-182 which includes pictures of facial hypertrophic scarring following curettage and electrodesiccation.

Cryosurgery has been used for many years by physicians and more recently by general consumers. Generally speaking, cryosurgical procedures include 1) the open probe methods where a porous applicator is filled with refrigerant and applied to the skin, 2) the closed probe method where the refrigerant circulates within a sealed applicator, cooling it, but does not contact the skin, and 3) the spray-on method which sprays the refrigerant onto the skin without the use of an applicator. U.S. Pat. No. 4,082,096, for example, describes these different methods. Open and closed probe cryosurgical products rely on the use of refrigerant liquids and/or gases to rapidly cool an applicator. Once cooled to an effective temperature, the applicator is applied to the skin for a period of seconds. The frozen tissue will necrose over a period of several weeks and slough from the body. New skin replaces the necrosed tissue.

Cryogenic methods that rely on the use of a cooled applicator rely on the principle of “heat of vaporization.” In an open probe method, as refrigerant is supplied to an applicator, the refrigerant will rapidly evaporate, cooling the applicator. Usually combinations of refrigerants are used to achieve optimal effects. Refrigerants known and used in the art and used alone or in combination include, but are not limited to, butane, propane and dimethyl ether.

The rate of evaporation dramatically influences the speed of cooling and the temperature reached. The speed of cooling is dramatically affected by the refrigerants used and the speed of evaporation from or within the applicator. In some cases, the effective temperature is not reached for 15-30 seconds. Users of such devices may not wait an appropriate time and therefore limit the effectiveness of the treatment. The effective temperature of devices may be reached for only a short time, thereby limiting the effective freezing of the target tissue. This is caused by the “heat-sink” effect. Local tissue is approximately 37° C. and accounts for the rapid warming of the cooled applicator. Since the freezing is a single application, the effectiveness of treatment is limited.

As an example of a cryogenic treatment, U.S. Pat. No. 5,330,745 issued to McDow discloses a method for cryogenically treating skin lesions optionally using a hollow fluid retaining device, such as a cone. According to the method, the hollow fluid retaining device (FIG. 2) is placed in an essentially upright position on a patient's skin in a manner whereby the bottom open end of the hollow fluid retaining device seals against the patient's skin entirely surrounding the skin lesion and the top open end of the hollow fluid retaining device is above the bottom open end. A liquid cryogenic agent is then introduced into the top open end of the upright fluid retaining device to cause formation of a liquid pool of the cryogenic agent to contact directly the entire area of the skin lesion. The hollow fluid retaining device is retained in the upright position surrounding the skin lesion after the introducing step has been terminated in order to retain the liquid pool of cryogenic agent within the bottom open end of the fluid retaining device and in contact directly with the skin lesion for a period of time to permit the cryogenic agent to reduce the temperature of the skin lesion to a temperature such that permanent, irreversible rupture of cellular membranes of cells of the skin lesion occurs while the cryogenic agent is evaporating. The fluid retaining device is subsequently removed from the upright position after the liquid cryogenic agent has evaporated. After the cryogenic agent has evaporated, the frozen skin tissue of the skin lesion is permitted to slowly thaw, preferably over a period of time that is at least about 45 to 65 seconds. U.S. Pat. No. 5,330,745 further discloses that it is often desirable and highly beneficial to permit the skin lesion to thaw completely after removing the fluid retaining device and to sequentially repeat the steps of the method described above. Such repetition of freeze-thaw cycles promotes increased cellular destruction and thus improved removal of skin lesions. U.S. Pat. No. 5,200,170, also to McDow, discloses a similar process.

In addition, there have been proposed ways of warming an applicator probe after using it as a refrigerated implement. For example, U.S. Pat. No. 3,502,081 discloses the use of electric resistance warming after a Joule-Thomson expansion to effect cooling. This technique is also disclosed in U.S. Pat. No. 3,901,241 in conjunction with evaporative cooling of a liquid refrigerant. This is cumbersome and expensive involving additional elements external to the probe.

In another type of cryosurgical instrument, e.g., disclosed in U.S. Pat. Nos. 3,548,829, 3,451,395, 3,393,679, 3,512,531, and 3,613,689, the cooling of the hollow working tip of the instrument is provided by unseating a flow valve element in the return line located downstream from the tip and simultaneously forming a restrictive orifice at the inlet to the tip by contacting an orifice seat with a moveable conduit. Refrigerant liquid or gas then flows from a source through the orifice and exhausts through the unseated downstream valve to effect cooling of the tip by evaporation or by a Joule-Thomson expansion. Warming of the tip is accomplished by seating the downstream valve element and simultaneously separating the moveable conduit from the orifice seat, thus permitting refrigerant at ambient temperature to flood the hollow working tip. This instrument type requires a moveable conduit and valving that are complex and therefore difficult and expensive to make on a consistently reliable basis.

U.S. Pat. Nos. 3,696,813 and 4,018,227 disclose warming by blocking the exhaust flow from the probe by closing a valve. Gas from the source, being rapidly increased in pressure would condense in the probe by reason of giving up latent heat, warming the probe. These patents also disclose a cryosurgical instrument cooled by the Joule-Thomson effect, and warmed by blocking off the exhaust so that there would be a quick rise of pressure within the probe. The pressure of the supplied gas builds up within the probe until it balances the source pressure, the rate of flow of the gas entering the tip cavity of the probe by its ordinary path (i.e., through the refrigerating restriction) decreasing as the pressure difference between the pressure source and the cavity sink (i.e., the probe cavity and its immediately adjacent passage volumes) diminishes; likewise the rate of flow from the source to the sink, which is subject to the control exercised by the restriction, similarly decreases.

U.S. Pat. Nos. 3,913,581, 3,782,386, Re. 28,657, and 4,146,030, all involve what is conveniently called “reverse flow” warming (as contrasted with “exhaust blocking” warming). Such constructions involve a provision in a Joule-Thomson type of instrument of a line of connection in which there is no deliberately provided restriction (but the volume, rate of flow of the gas is controlled or restricted in the freeze mode to undergo the Joule-Thomson expansion) between a source of pressurized gas and the cavity of a cryosurgical probe, so that the operator can operate valve means so as to admit warming gas at substantially ambient temperature at a high rate, into the cavity, usually through the unrestricted exhaust conduit or line (hence, the nomenclature “reverse-flow”), where such gas performs the required warming. The gas performs the warming largely or partially by condensing within the probe, the condensate then being allowed to escape as a liquid, or partly as a liquid and partly as a gas, being purged to atmosphere by following or entraining gas, the probe temperature having been raised by the latent heat. Liquid condensate remaining within the probe may be purged by venting or subsequently to warming, during the early part of the next cooling mode. While effective to provide a quick warming mode in small, e.g., ophthalmic instruments, in instruments adapted to operate on a larger scale, however, wherein by virtue of higher refrigerant flow through larger volumes, cooling of the exhaust passage (including exhaust valves) is greater, liquid condensate is likely to form preferentially in the exhaust rather than within the cavity, and may subsequently flow into the cavity, this leading to partial failure to warm or unacceptably slow warming. Finally, such instruments usually have hand- or foot-operated valves separate from the instrument itself. Consequently, they require separate consoles and extra high-pressure hose lines making the overall system more expensive, bulky and difficult to move about.

In addition, U.S. Pat. No. 3,536,075 discloses that cooling may be effected by evaporation of liquid refrigerant ducted to the tip, rather than a Joule-Thomson, isoentropic expansion of a refrigerant gas. This necessitates connecting the common exhaust line to a vacuum to remove the boiled gas and warm defrost liquid used to flood the tip, which would be inoperative in a Joule-Thomson instrument as insufficient back pressure of the gas would be generated, if the gas was sucked from the exhaust, to warm and defrost the tip after an isoentropic expansion of the gas.

U.S. Pat. Nos. 3,933,156 and 4,015,606 are specific to constructions seeking to minimize some of the problems noted with respect to the prior art instruments. For example, U.S. Pat. No. 3,933,156 discloses insulating outer tubes surrounding concentric expansion tubes (i.e., an inlet and an exhaust tube) to minimize heat transfer between the tubes and condensation of refrigerant in the line of the process. In U.S. Pat. No. 4,015,606 discloses a cryosurgical instrument having a construction for controlling the freeze zone in the tip by moving the exhaust tube conduit in the tip towards or away from the supply (inlet) tube conduit.

In addition, U.S. Pat. No. 4,377,168 discloses cryosurgical instruments in which a hollow probe tip is cooled by the passage of a fluid refrigerant through the hollow interior of the tip. In particular, the invention is concerned with instruments cooled by the Joule-Thomson effect or isenthalpic expansion of a gaseous refrigerant through a flow restriction adjacent the hollow interior or cavity of the tip. In the Joule-Thomson system, cooling is the result of the refrigerant fluid suffering a drop of pressure caused by the flow restriction through which the fluid passes. The restriction and consequently the cooling takes place within the instrument itself, and therefore the refrigerant reaches the tip of the instrument from the source at, or substantially at, the source pressure and temperature and the cooling occurs in the immediate vicinity of the tip. The refrigerant occupies the tip cavity of the instrument as a cold gaseous fluid cooled by isenthalpic gaseous expansion and takes up its latent heat of vaporization from the wall of the tip cavity and therefore from the tissue with which the probe is contacted. The cold gaseous fluid may carry with it some proportion of liquid refrigerant in the form of droplets or mist. It is usually important that such a cryosurgical instrument be quickly and precisely controlled in the cooling, refrigeration, or “freezing” phase; while it is of comparable importance to control warming, defrosting, (or “thawing”) of the instrument, particularly because it is desirable that it can quickly be released from the congelation with the tissue which it effects. Such warming involves a release or supply of heat, such as to raise the temperature of the probe and thereby the tissue and whatever other material constitutes their interface, such as saline solution deliberately used at such interface. The warming here functions to facilitate the separation between the probe and the tissue upon completion of the treatment.

Finally, it has been suggested to use freeze/thaw cycles on human primary prostatic adenocarcinoma cells. See Tatsutani, Krinstine; Rubinsky, Boris; Onik, Gary; and Dahiya, Rajvir, “Effect of Thermal Variables on Frozen Human Primary Prostatic Adenocarcinoma Cells”, Adult Urology, pgs. 441-447, 1996. Accoriding to Tatsutani, et al., a double freeze/thaw cycle is required to ensure complete cell destruction at high subzero temperatures. This article discusses the utility of prostate cryosurgery, among others.

Accordingly, there is a need for an improved cryosurgical device and method capable of completely freezing a target area to kill all localized cells. This invention meets that need by providing a device and modified spray-on method for cycling between cold and warm to more effectively kill all of the localized cells in the area targeted for treatment.

### SUMMARY OF THE INVENTION

Accordingly, the invention relates to a method of freezing a lesion on biological tissue, and a cryosurgical probe adapted to facilitate the freezing of a lesion on biological tissue, and a cryosurgical instrument operable to freeze a lesion on biological tissue.

According to the method, a cryosurgical probe is positioned against the lesion. The spray-on type cryosurgical probe includes a receiving end adapted to receive a refrigerant, a tissue contact end including an orifice, and a hollow portion including at least one exhaust port, wherein the orifice is adapted to expose at least a portion of the lesion to the refrigerant, and the hollow portion extends from the receiving end to the tissue contact end, and is adapted to allow the flow of refrigerant from the receiving end to the tissue contact end. The tissue contact end also preferably includes a guard portion adapted to prevent excessive exposure of the biological tissue surrounding the lesion to the refrigerant. Then, a batch of a liquid refrigerant is dispensed (i.e. sprayed) into the cryosurgical probe such that at least a portion of the refrigerant is in direct contact with the tissue via the orifice. After the dispensing step, the tissue contact end is maintained in contact with the tissue for a sufficient amount of time to allow the refrigerant to evaporate completely (i.e. a freezing step) and the tissue to at least partially thaw (i.e. a thawing step). During the dispensing and maintaining steps, at least a portion of the refrigerant evaporates, for example, over a period of 0.1 to 200 seconds, with the gaseous, evaporated, refrigerant exiting through the exhaust port(s). The duration of the maintaining step varies depending on the depth and size of the lesion. The dispensing and maintaining steps may be repeated at least once to ensure a more complete destruction of the lesion. The conditions under which the steps are repeated may be based on one or more operating conditions. The operating conditions may be based on evidence previously gathered for the type of lesion being treated. For example, certain types of lesions may require more cycles of freeze/thaw than others.

The invention further relates to a cryosurgical probe adapted to be positioned on an end of a cryosurgical instrument, the cryosurgical probe being further adapted to receive a refrigerant to facilitate the freezing of a lesion on biological tissue via the evaporation of the refrigerant. The cryosurgical probe includes a receiving end, a hollow portion, and a tissue contact end. The receiving end is adapted to receive the refrigerant. The tissue contact end includes an orifice adapted to expose at least a portion of the lesion to the refrigerant. The hollow portion extends from the receiving end to the tissue contact end, and is adapted to allow the flow of refrigerant from the receiving end to the tissue contact end. The hollow portion further includes at least one exhaust port.

The cryosurgical probe is preferably removable from the cryosurgical instrument. This permits the use of disposable cryosurgical probes to be used for individual treatments on a single cryosurgical device. The tissue contact end preferably includes a guard portion adapted to prevent excessive exposure of the biological tissue surrounding the lesion to the refrigerant. Also, the receiving end may be adapted to receive the refrigerant from a refrigerant supply. The shape of the orifice on the tissue contact end may vary, but is preferably substantially the same as the shape of the lesion. The shape of the orifice may also be substantially circular, oblong, or oval.

The invention also relates to a cryosurgical instrument operable to freeze a lesion on biological tissue. The cryosurgical instrument includes a cryosurgical probe, a refrigerant supply, and a control means. The cryosurgical probe, which may be removable from the cryosurgical instrument, is adapted to receive a refrigerant to facilitate the freezing of the lesion on the biological tissue. Also, the cryosurgical probe preferably includes a receiving end adapted to receive the refrigerant, a tissue contact end including an orifice and a guard portion, and a hollow portion including at least one exhaust port, wherein the orifice is adapted to expose at least a portion of the lesion to the refrigerant, the guard portion is adapted to prevent excessive exposure of the biological tissue surrounding the lesion to the refrigerant, and the hollow portion extends from the receiving end to the tissue contact end, and is adapted to allow the flow of refrigerant from the receiving end to the tissue contact end. The refrigerant supply is in communication with the cryosurgical probe, and is adapted to provide the cryosurgical probe with refrigerant. The control means is for controlling the supply of refrigerant from the refrigerant supply to the cryosurgical probe. The control means may control the supply of the refrigerant to the cryosurgical probe based on one or more operating conditions.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIGS. 1A-1I illustrate exemplary cryosurgical probes of the invention.

FIG. 2 is a flowchart of an exemplary method of the invention.

FIG. 3 illustrates an exemplary cryosurgical instrument of the invention.

FIG. 4 illustrates an alternate, magnified view, of the cryosurgical instrument of FIG. 3.

FIG. 5 illustrates an exemplary user interface that may be used to control a cryosurgical instrument of the invention.

### DETAILED DESCRIPTION OF THE INVENTION

As described above, the invention relates to a spray-on cryosurgical probe, a method of freezing a lesion on biological tissue, and a cryosurgical instrument operable to freeze a lesion on biological tissue. Generally, cryosurgical treatment of skin lesions is affected by the freezing of the afflicted tissue in three dimensions. In order to achieve effective cryosurgical treatment, the afflicted tissue and lesion must be completely frozen at some point. In accordance with the invention, treatment effectiveness may be increased further by exposing the tissue to the cryogen more than once. Living cells receive damage from cryosurgical treatment both during freezing and thawing steps. Some cells may survive a single freezing treatment, but their fraction reduces significantly with subsequent freezing and thawing cycles. In addition, the cycling allows for a more complete control over the depth and degree of freezing, as the duration of the cycling may be stopped whenever the treatment is completed.

Accordingly, the invention utilizes a cycling of freezing and thawing stages to deliver optimal therapy. This result is made possible through a controlled delivery of a liquid refrigerant to the target tissue followed by a controlled thawing. The cycling of cold from the cryogen and heat from the surrounding skin allows the increase destruction of the target tissue to the desired depth and width while maintaining control over the process and substantially preventing unnecessary freezing of healthy tissue. Using this method and the instrument described herein, extremely low temperatures, for example, −90° C. (using nitrous oxide as the refrigerant), may be applied to the target areas of the skin. Operators will be able to select the number of bursts of gas to allow rapid freezing and thawing of the area for maximal effect.

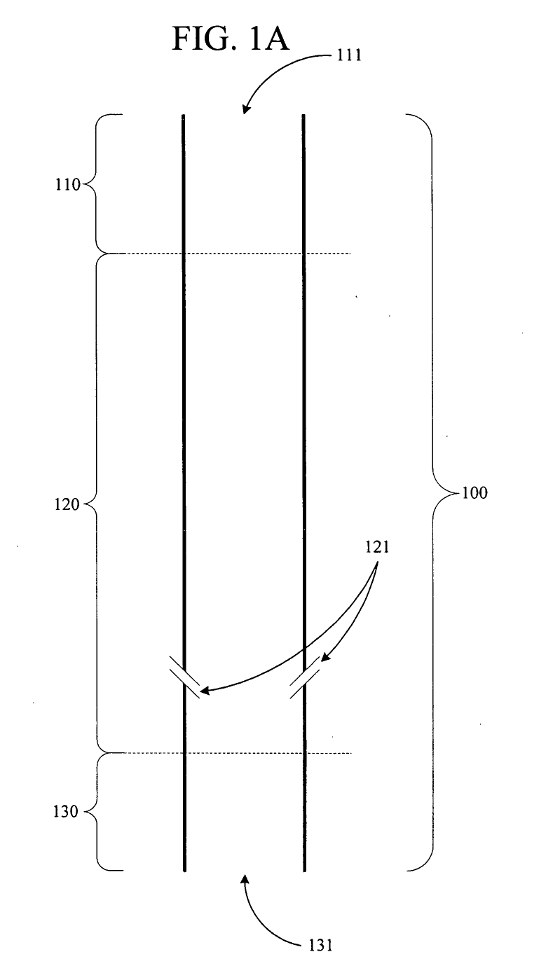

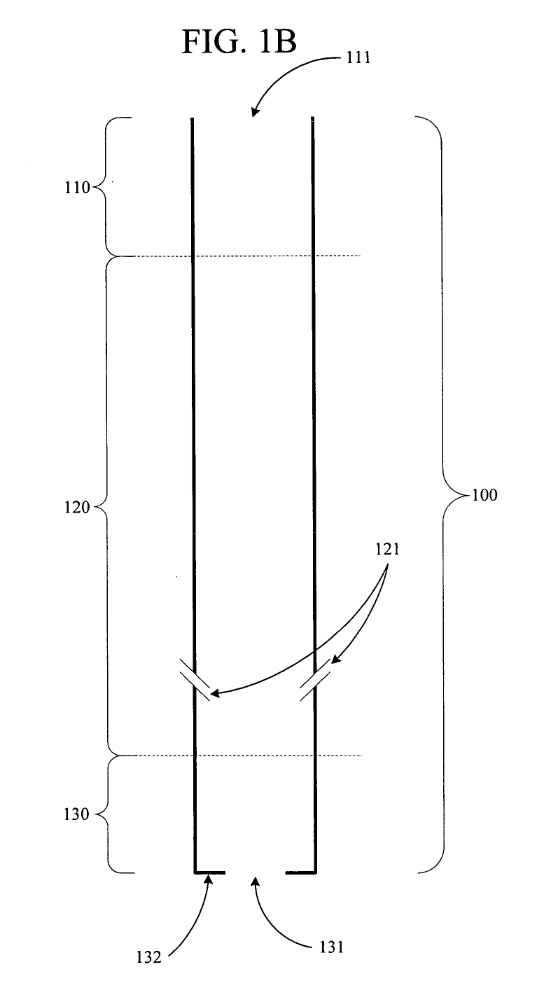

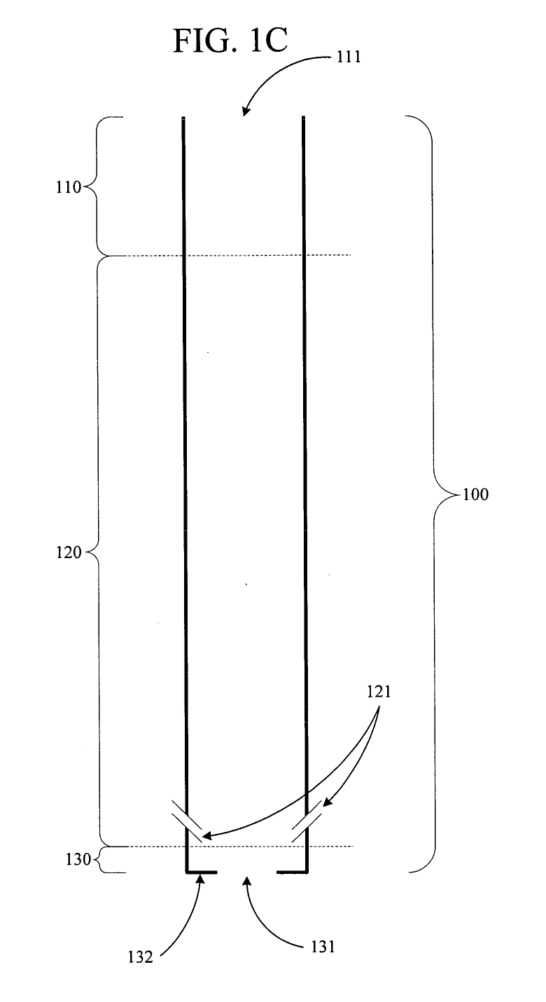

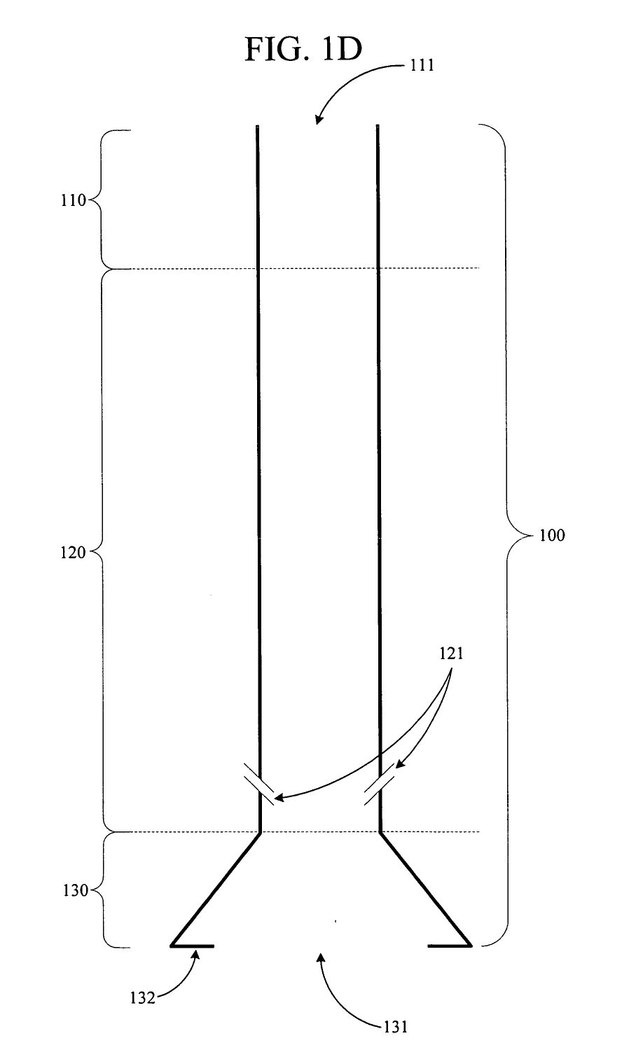

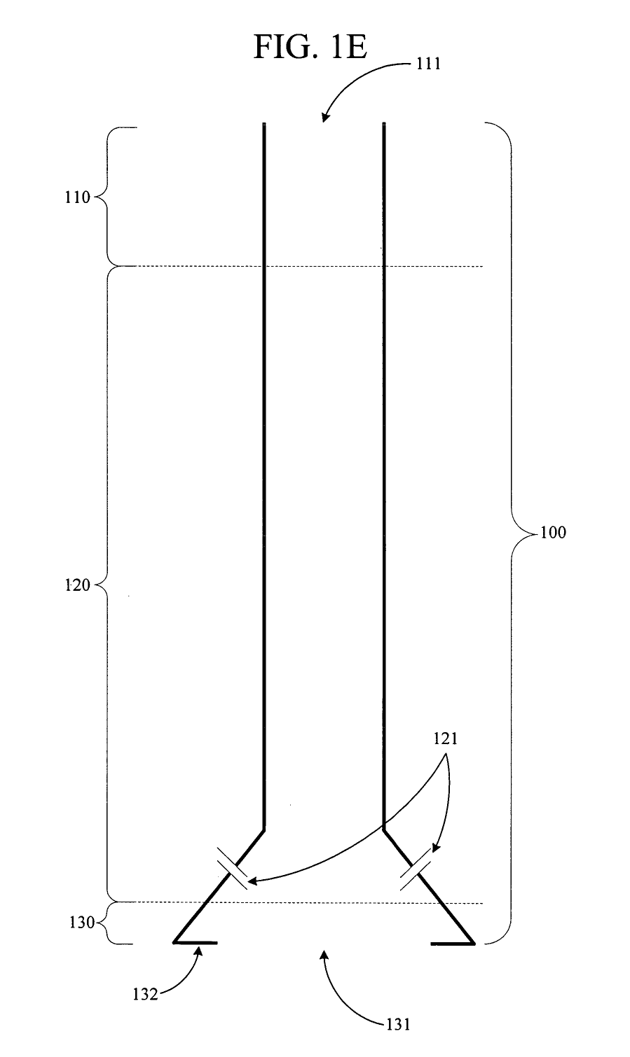

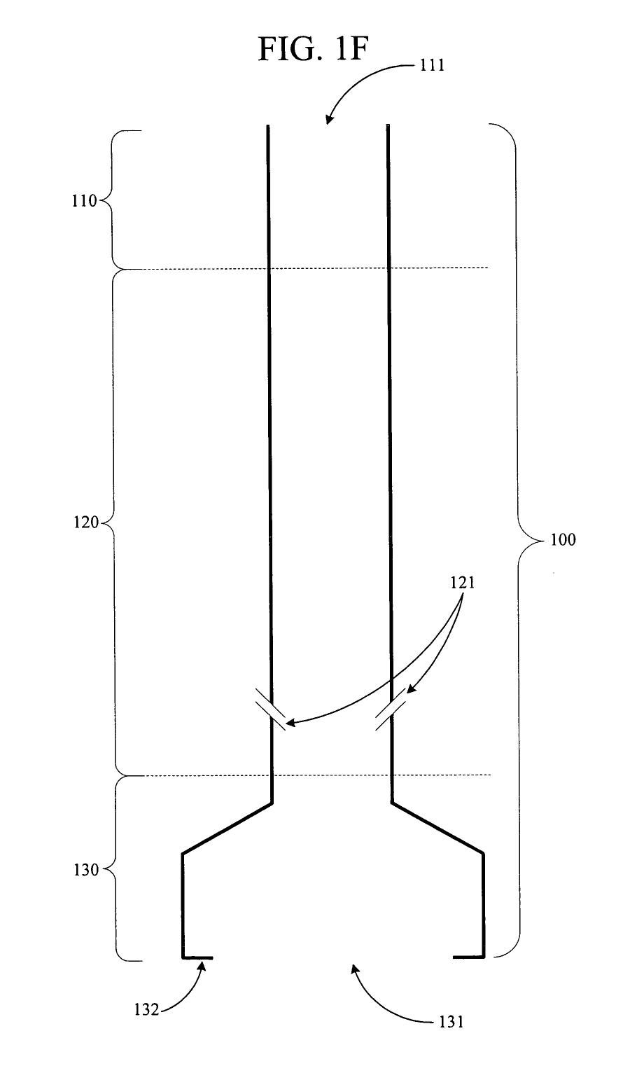

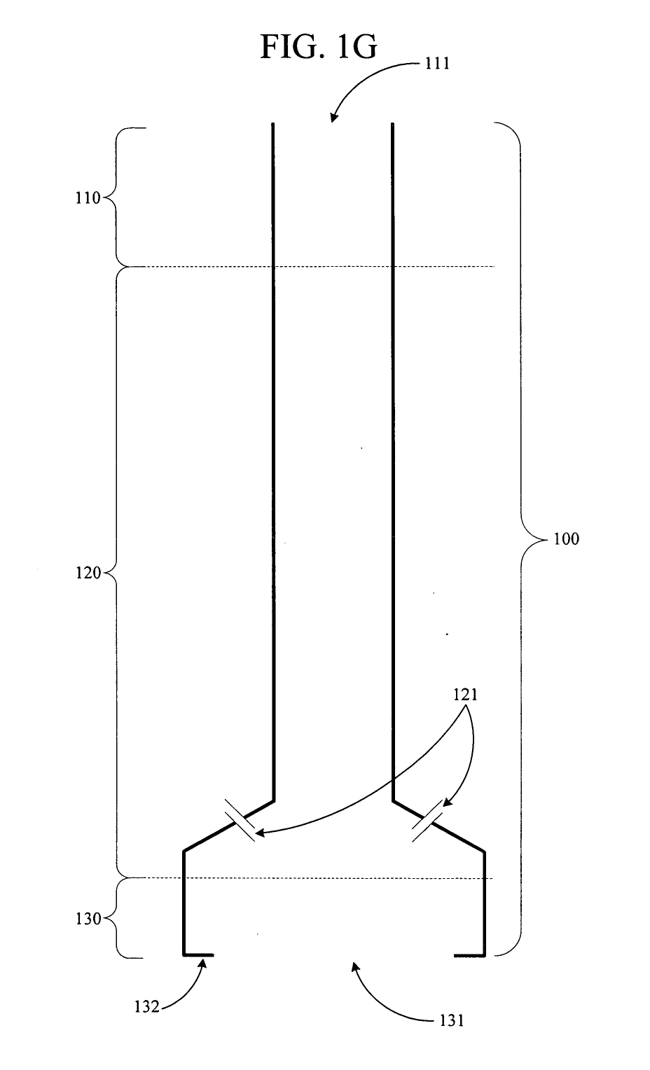

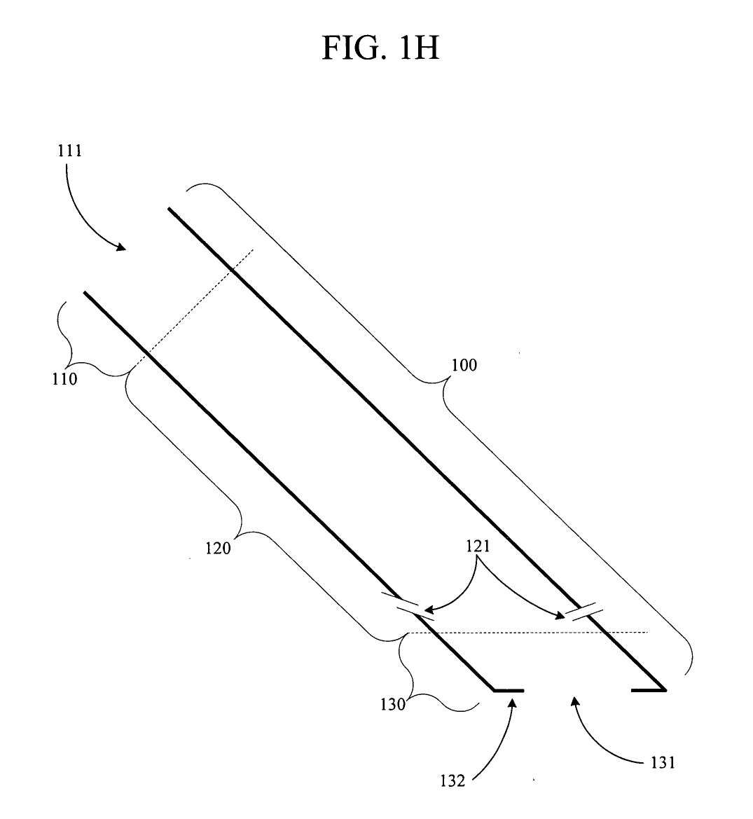

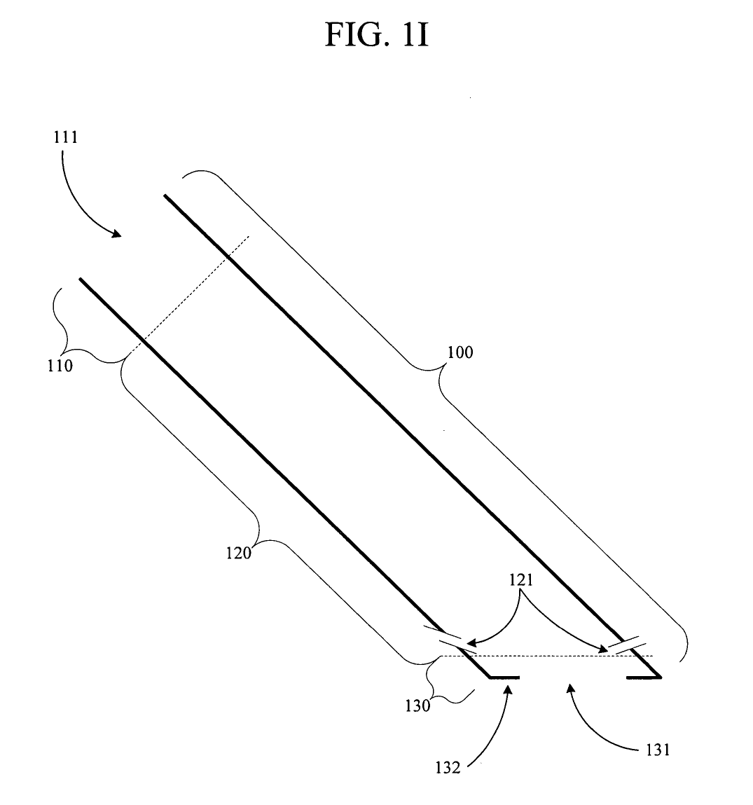

FIGS. 1A-1I each illustrate an exemplary cryosurgical probe 100 of the invention. In particular, cryosurgical probe 100 is adapted to be positioned on an end of a cryosurgical instrument. Cryosurgical probe 100 is also adapted receive a refrigerant to facilitate the freezing of a lesion on biological tissue via the evaporation of the refrigerant.

Cryosurgical probe 100 includes a receiving end 110, a hollow portion 120, and a tissue contact end 130. Receiving end 110 is adapted to receive the refrigerant via entrance 111 from any suitable refrigerant supply. For example, suitable refrigerant supplies include traditional liquid gas storage container or tanks, custom made cartridges or stationary, large tanks. In addition, cryosurgical probe 100 is preferably removable from the cryosurgical instrument. In this regard, receiving end 110 also preferably serves as a connector to the cryosurgical instrument and is sized appropriately to engage the cryosurgical instrument. This engagement may be a threaded engagement, a frictional engagement, a click-type snapping engagement, or the like. In addition, the receiving end may be either the internal or external portion of the connection to the cryosurgical device. Furthermore, the receiving end may be configured to fit any existing cryosurgical instruments capable of dispensing refrigerant.

Also, any refrigerant, or cryogen, known in the cryosurgical arts may be used provided that the refrigerant is capable of rapid evaporation resulting in a rapid decrease in temperature in a localized zone. For example, liquid gases such as nitrous oxide, CO₂, dimethyl ether, propane, butane, liquid nitrogen, and combination thereof, may be used.

Tissue contact end 130 includes an orifice 131, which is adapted to expose at least a portion of the lesion to the refrigerant. The shape of orifice 131 on tissue contact end 130 may vary. However, orifice 131 is preferably sized and substantially shaped the same as the lesion. For example, orifice 131 may have an internal diameter of 3-8 mm, depending on the size of the lesion. Larger sizes may be used for larger lesions, or as treatment conditions require. In addition, the shape of orifice 131 may be, for example, circular, oblong, or oval. Furthermore, the tissue contact end 130 may be formed of any suitable non-stick material suitable for use in medical devices, such as plastic, or any suitable metals, glasses, or composites.

In addition, as is shown in FIGS. 1A-1I, tissue contact end 130 also includes an optional guard portion 132, which is adapted to prevent excessive exposure of the biological tissue surrounding the lesion to the refrigerant. The primary function of guard portion 132 is to protect the tissue surrounding the lesion being treated. When tissue contact end 130 includes guard portion 132, it is preferred that tissue contact end 130 be formed of a material that is non-conductive with respect to heat to prevent damage to the tissue under guard portion 132.

Hollow portion 120 extends from receiving end 110 to tissue contact end 130, and is adapted to allow the flow of refrigerant from receiving end 110 to tissue contact end 130. The characteristics of the hollow portion (i.e. size, material) are preferably based on the dimensions and materials of the orifice and the receiving end, and the length of the hollow portion will be optimized experimentally to provide uniform delivery of the refrigerant. Hollow portion 120 further includes at least one exhaust port 121. Each exhaust port 121 functions to provide an avenue for orderly escape of evaporated cryogen and to divert the flow of evaporated gas away from the skin, the cryosurgical instrument, and the operator's hands. FIGS. 1A-1I illustrate exemplary cryosurgical probes utilizing a pair of exhaust ports 121. After the refrigerant is dispensed into tissue contact end 130, the refrigerant rapidly evaporates, and the resulting gases rapidly exit cryosurgical probe 100 via exhaust ports 121. The number and size of exhaust ports 121 impact the rate of evaporation of the dispensed refrigerant and assist in controlling freeze to thaw cycle times. In addition, each exhaust port 121 should be positioned in hollow portion 120 above the maximum level of the dispensed liquid refrigerant to prevent spillage of the refrigerant out of cryosurgical probe 100 prior to evaporation. Thus, exhaust ports 121 may be located at any height above tissue contact end 130 as long as they are far enough above tissue contact end 130 to allow sufficient refrigerant to be used.

While receiving end 110 should be sized appropriately to receive the refrigerant from the refrigerant supply, a specific cross-sectional geometry of the receiving end and the hollow portion is not required. For example, as long as their functional requirements are satisfied, the cross-sectional shape of receiving end 110 and hollow portion 120 may be any geometry, including circular, oblong, square, rectangular, or any other geometry.

Furthermore, the size and shape of tissue contact end 130 may vary greatly depending on the size and type of lesion to be treated. FIGS. 1A-1I illustrate various exemplary shapes and sizes of hollow portion 120 and tissue contact end 130 including different dimensions for orifice 131 and optional guard portion 132. For example, a larger tissue contact end may be used when the lesion is large. Of course, more refrigerant may be dispensed into a larger tissue contact end than a smaller tissue contact end. There is no specific requirement for the shape of the tissue contact end except to ensure that the orifice can be sized appropriately to treat the lesion. For example, the cross-sectional geometry of the tissue contact end may be circular, oblong, square, rectangular, or any other suitable shape. In addition, as described above, the positions of exhaust ports 121 may vary with respect to their proximity to tissue contact end 130. Different possible exemplary positions of exhaust ports 121 are also shown in FIGS. 1A-1I. Finally, it may be preferred to position the cryosurgical probe at an angle relative to the tissue, for example, for ergonomic comfort. In this respect, FIGS. 1H and 1I illustrate cryosurgical probes 100 having an angled tissue contact end 130. When this embodiment is used, each of exhaust ports 121 are preferably equidistant, from a perpendicular perspective, from tissue contact end 130. This positioning allows for the rapid escape of the gaseous refrigerant after evaporation without having one exhaust port 121 being closer to the surface of the refrigerant than another after the refrigerant is dispensed into tissue contact end 130. In addition, the exact positioning of the exhaust ports on the hollow portion may be adjusted for each different probe size to facilitate better performance of the two main functions of the exhaust ports, namely, to provide orderly escape for evaporated cryogen, and to divert the flow of the evaporated cryogen away from the skin, the cryosurgical instrument and the operator hands.

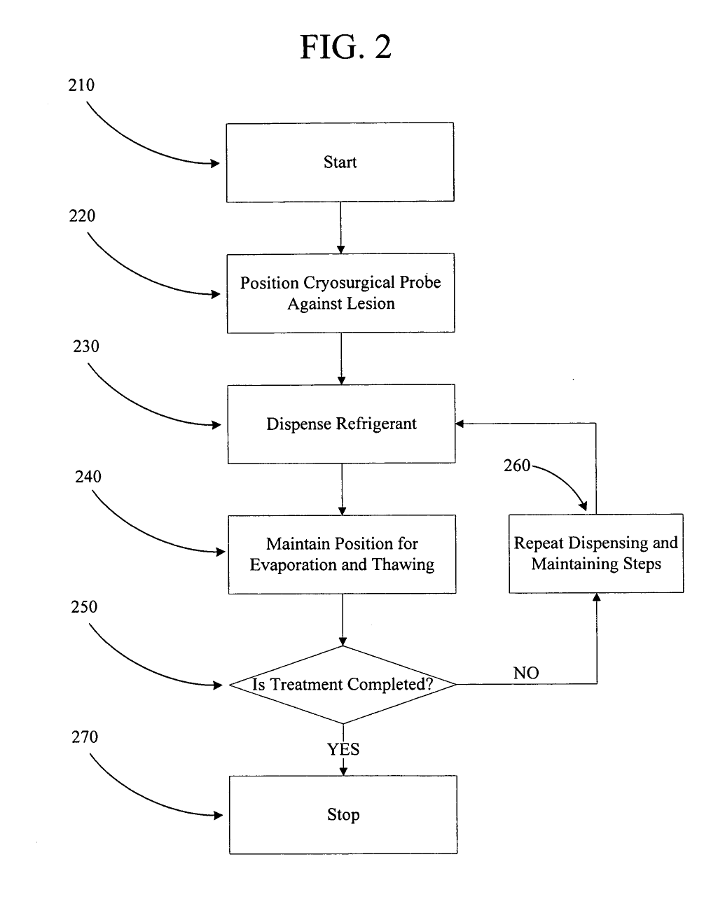

FIG. 2 is a flowchart of an exemplary method of the invention for freezing a lesion on biological tissue. After a treatment session is started at step 210, the tissue contact end of the cryosurgical probe is positioned against the lesion to be treated in step 220. As described above with reference to FIGS. 1A-1I, the cryosurgical probe preferably includes a receiving end, a hollow portion, and a tissue contact end. The receiving end is adapted to receive the refrigerant, the tissue contact end includes an orifice, and the hollow portion includes at least one exhaust port. The orifice is adapted to expose at least a portion of the lesion to the refrigerant, and the hollow portion extends from the receiving end to the tissue contact end, and is adapted to allow the flow of refrigerant from the receiving end to the tissue contact end. When properly positioned, at least a portion of the lesion should be positioned within the orifice on the tissue contact end to allow direct contact between a liquid refrigerant within the cryosurgical probe and the lesion. As is described above, it is also preferred that the tissue contact end include a guard portion, which is adapted to prevent excessive exposure of the biological tissue surrounding the lesion to the refrigerant

In step 230, a batch of a refrigerant is dispensed into the cryosurgical probe. As described above, because the lesion is positioned within the orifice of the tissue contact end, at least a portion of the refrigerant is in direct contact with the tissue via the orifice. The amount of refrigerant dispensed may vary depending on the characteristics of the lesion. For example, a larger lesion may require more refrigerant than a smaller lesion. Furthermore, the refrigerant may be dispensed instantaneously, or may be slowly dispensed over a period of time, for example, 0.1 to 15 seconds. During the dispensing step, the liquid refrigerant comes into contact with the skin and undergoes a rapid evaporation, which causes the temperature to drop within the cryosurgical probe, thereby freezing the portion of the tissue exposed within the orifice. The vapors from the evaporation exit the cryosurgical probe via the exhaust ports in the hollow portion. The rate of the evaporation of the refrigerant varies depending on which refrigerant is used. Any refrigerant or combination of refrigerants known for use in cryosurgery may be used with the cryosurgical probe and method of this invention.

After the dispensing step is completed, the treatment undergoes a maintaining step 240 in which the tissue contact end is maintained in contact with the tissue for a sufficient amount of time to allow any refrigerant present in the cryosurgical probe to evaporate completely and the tissue to at least partially thaw. The duration of this thawing step can vary widely depending on the properties of the surrounding tissue, the size of the lesion, and the refrigerant used, but is preferred to be around 0.1 to 40 seconds.

After the maintaining step 240, during which the refrigerant is evaporated followed by thawing of the skin, a determination can be made regarding whether the treatment is complete (Step 250). This determination can be made based on predetermined criteria, or can be made during the procedure by the person or persons administering the treatment. As an alternative, this determination can be made by a programmed computing device in accordance with conditions set forth in software or stored in the computing device, or conditions obtained during the procedure. In this respect it is possible to utilize the methods of the invention with a computerized system that allows for automatic control of the cryosurgical probe and metering of the refrigerant to the cryosurgical probe. This can be made possible through the use of electronically controlled valves, such as solenoid valves, to meter the supply of refrigerant to the cryosurgical probe.

Some types of lesions may require, as an example, 5 to 10 cycles of freezing and thawing steps to completely treat and remove the lesion. Others may require more or less cycles. During this process, the repeating of step 230 and step 240 provides the ability to cycle from freeze to thaw while treating an area. This is made possible by maintaining the cryosurgical probe in a position through the treatment and repeated cycles. This allows a controlled delivery of refrigerant to occur, thereby facilitating increased efficacy. The conditions under which the steps are repeated may be based on one or more operating conditions, such as the type of lesion being treated.

If it is determined in step 250 that the treatment is not complete, step 260 provides that step 230 and step 240 should be repeated. After the theses steps are repeated, it is again determined whether the treatment is complete in step 250. If so, Stop step 270 represents the end of the treatment.

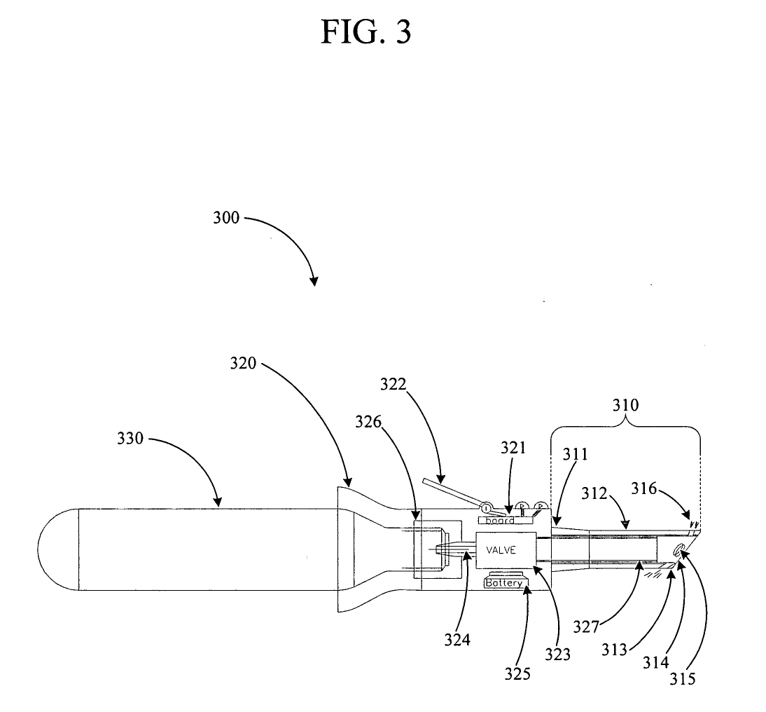

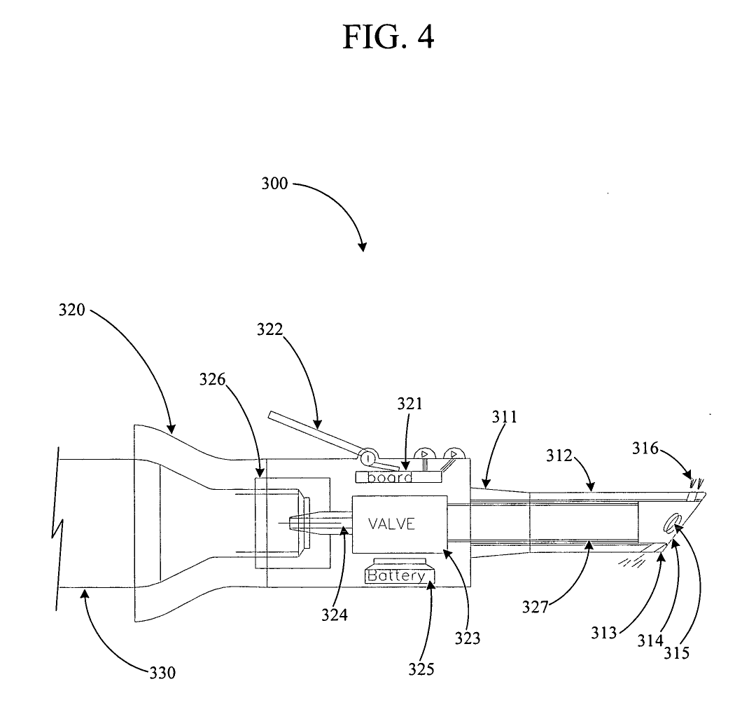

FIG. 3 illustrates an exemplary cryosurgical instrument of the invention, and FIG. 4 illustrates an alternate, magnified view, of the cryosurgical instrument of FIG. 3. In particular, the cryosurgical instrument is operable to freeze a lesion on biological tissue. Cryosurgical instrument 300 includes a cryosurgical probe 310, a refrigerant supply 330, and a control means 320. Cryosurgical probe 310, which may be removable from cryosurgical instrument 300, is adapted to receive a refrigerant from refrigerant supply 330 to facilitate the freezing of the lesion on the biological tissue. Generally, cryosurgical probe 310 is a contact cone or receiver that constricts the flow of the refrigerant to a directed location with exhaust port(s) to allow rapid controlled escape of the gas(es).

In addition, cryosurgical probe 310 preferably includes a receiving end 311 adapted to receive the refrigerant from refrigerant supply 330, a tissue contact end 313, and a hollow portion 312, which includes at least one exhaust port 316. Tissue contact end 313 also includes an orifice 315, which is preferably adapted to expose at least a portion of the lesion to the refrigerant received from refrigerant supply 330. Hollow portion 312 extends from receiving end 311 to tissue contact end 313, and is adapted to allow the flow of refrigerant from receiving end 311 to tissue contact end 313. As described above, cryosurgical probe 310 also includes one or more exhaust ports 316 which allow for rapid controlled escape of the gas(s) upon evaporation, and it is preferred that tissue contact end 313 includes a guard portion 314, which is adapted to prevent excessive exposure of the biological tissue surrounding the lesion to the refrigerant.

Refrigerant supply 330 is in communication with cryosurgical probe 310 via control means 320, and is adapted to provide cryosurgical probe 310 with refrigerant. Control means 320 controls the supply of refrigerant from refrigerant supply 330 to cryosurgical probe 310. Control means 320 may control the supply of the refrigerant to cryosurgical probe 310 based on one or more operating conditions. As is illustrated in FIG. 3, refrigerant supply 330 is preferably a gas cylinder or container with the liquefied or compressed refrigerant gas(es). Any suitable container may be used, or optionally, the refrigerant may be supplied from a remote refrigerant supply that is maintained in communication with the cryosurgical probe.

In addition, cryosurgical probe 310 may be connected to control means 320 via an extended connector, such as flexible tubing. In this situation, it is preferred that the cryosurgical probe include a handle portion or other element (not shown) that allows the treatment provider to control the position of the cryosurgical probe without necessarily holding the remaining components of the cryosurgical instrument. In this manner, the cryosurgical probe may be controlled directly, with the refrigerant being supplied from a remote refrigerant supply via the flexible tubing, thereby increasing the comfort to the treatment provider due to the decreased weight and size of the applicator.

Control means 320 may be any suitable control means having any suitable components that enable controlled dispensing of the refrigerant. For example, as is shown in FIG. 3, control means 320 may include a control lever 322, a circuit board 321, a battery 325, a valve 323, a connection assembly 326, a refrigerant passage 324, and a dispensing tip 327. Control lever 322 allows metering of the refrigerant through the valve. Dispensing tip 327 facilitates the flow of refrigerant from refrigerant supply 330 through control means 320 and directs the refrigerant towards tissue contact end 313.

During operation of the cryosurgical instrument illustrated in FIGS. 3 and 4, when control lever 322 is depressed, refrigerant flows from refrigerant supply 330 through control means 320 via refrigerant passage 324, exits control means 320 via dispensing tip 327, and pools at tissue contact 313 in direct contact with the lesion or tissue exposed via orifice 315. When a sufficient amount of refrigerant has been dispensed, control lever 322 is released, the flow of refrigerant from refrigerant supply 330 is stopped, and the refrigerant pooled in tissue contact end 313 is allowed to evaporate. At this point, the dispensed refrigerant may be a liquid or a mixture of liquid and gas. As the refrigerant warms and evaporates, freezing the skin, the resulting vapors exit cryosurgical probe 310 via exhaust ports 316. This process may be repeated as needed.

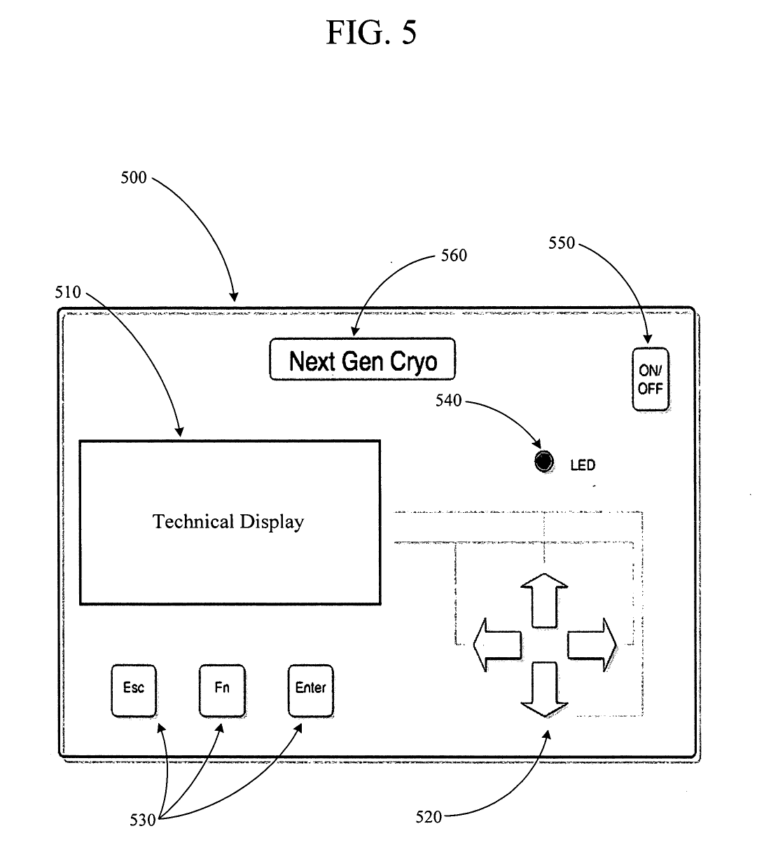

FIG. 5 illustrates an exemplary user interface that may be used to control a cryosurgical instrument of the invention. A person of ordinary skill in the art will readily appreciate the wide variety of user interfaces that are suitable for use with medical devices such as the cryosurgical instrument described herein. As illustrated in FIG. 5, exemplary user interface 500 includes a technical display 510, directional buttons 520, controls 530, and LED indicator 540, power switch 550, and title display 560. Technical display 510 includes information pertinent to the procedure such as information regarding the time or duration of the procedure, the status of the cryosurgical instrument, and the characteristics of the refrigerant supply, the cryosurgical instrument, the cryosurgical probe, and the lesion to be treated. The types of information displayed may vary widely depending on what information is desired. In addition, various types of information may be input from the user as appropriate. Directional buttons 520 may be used to navigate the information displayed on technical display 510, for example, through menus. Controls 530 may be used to select specific items displayed on technical display 510, or to otherwise navigate through technical display 510 or control the cryosurgical instrument. LED indicator 540 can be used to visually indicate any condition related to the cryosurgical instrument, the refrigerant supply, or the cryosurgical probe, or the user interface. For example, LED indicator 540 may light up to indicate that the supply of refrigerant in refrigerant supply is running low, or that the refrigerant supply is not properly connected to the control means. In addition, LED indicator may be used to indicate that the cryosurgical probe is not installed properly, or has already been used. LED indicator may also be used to indicate any other reaction condition.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A method of freezing a lesion on biological tissue comprising the steps of:
1) positioning a cryosurgical probe against the lesion, the cryosurgical probe including a receiving end adapted to receive a refrigerant, a tissue contact end including an orifice, and a hollow portion including at least one exhaust port, wherein the orifice is adapted to expose at least a portion of the lesion to the refrigerant, and the hollow portion extends from the receiving end to the tissue contact end, and is adapted to allow the flow of refrigerant from the receiving end to the tissue contact end;
2) dispensing a batch of a refrigerant into the cryosurgical probe such that at least a portion of the refrigerant is in direct contact with the tissue via the orifice;
3) maintaining the tissue contact end in contact with the tissue for a sufficient amount of time to allow the refrigerant to evaporate completely and the tissue to at least partially thaw; and
4) repeating steps 2 and 3 at least once to ensure a more complete destruction of the lesion.

2. The method of claim 1, wherein steps 2 and 3 are repeated in accordance with one or more operating conditions.

3. The method of claim 1, wherein the refrigerant is dispensed into the cryosurgical probe in step 2 for a period of 0.1 to 15 seconds.

4. The method of claim 1, wherein the maintaining step in step 3 occurs for a period of 0.1 to 40 seconds.

5. The method of claim 1, wherein the refrigerant is selected from the group consisting of nitrous oxide, CO₂, dimethyl ether, propane, butane, liquid nitrogen, and combinations thereof.

6. The method of claim 1, wherein the tissue contact end further comprises a guard portion adapted to prevent excessive exposure of the biological tissue surrounding the lesion to the refrigerant.

7. A cryosurgical probe adapted to be positioned on an end of a cryosurgical instrument, the cryosurgical probe being further adapted to receive a refrigerant to facilitate the freezing of a lesion on biological tissue via the evaporation of the refrigerant comprising:
a receiving end adapted to receive the refrigerant;
a tissue contact end including an orifice adapted to expose at least a portion of the lesion to the refrigerant and
a hollow portion including at least one exhaust port, wherein the hollow portion extends from the receiving end to the tissue contact end, and the hollow portion is adapted to allow the flow of refrigerant from the receiving end to the tissue contact end.

8. The cryosurgical probe of claim 7, wherein the shape of the orifice is substantially the same as the shape of the lesion.

9. The cryosurgical probe of claim 7, wherein the shape of the orifice is substantially circular, oblong, oval, or any other suitable geometric shape.

10. The cryosurgical probe of claim 7, wherein the hollow portion includes at least two exhaust ports.

11. The cryosurgical probe of claim 7, wherein the cryosurgical probe is removable from the cryosurgical instrument.

12. The cryosurgical probe of claim 7, wherein at least the tissue contact end is formed of at least one of plastic, metal, glass, or a composite material.

13. The cryosurgical probe of claim 7, wherein the receiving end is adapted to receive the refrigerant from a remotely located refrigerant supply.

14. The cryosurgical probe of claim 7, wherein the tissue contact end further comprises a guard portion adapted to prevent excessive exposure of the biological tissue surrounding the lesion to the refrigerant.

15. A cryosurgical instrument operable to freeze a lesion on biological tissue comprising:
a cryosurgical probe adapted to receive a refrigerant to facilitate the freezing of the lesion on the biological tissue, the cryosurgical probe including a receiving end adapted to receive the refrigerant, a tissue contact end including an orifice, and a hollow portion including at least one exhaust port, wherein the orifice is adapted to expose at least a portion of the lesion to the refrigerant, and the hollow portion extends from the receiving end to the tissue contact end, and is adapted to allow the flow of refrigerant from the receiving end to the tissue contact end;
a refrigerant supply in communication with the cryosurgical probe, the refrigerant supply being adapted to provide the cryosurgical probe with refrigerant; and
a control means for controlling the supply of refrigerant from the refrigerant supply to the cryosurgical probe.

16. The cryosurgical instrument of claim 15, wherein the control means controls the supply of the refrigerant to the cryosurgical probe based on one or more operating conditions.

17. The cryosurgical instrument of claim 15, wherein the cryosurgical probe is removable from the cryosurgical instrument.

18. The cryosurgical instrument of claim 15, wherein the refrigerant is selected from the group consisting of nitrous oxide, CO₂, dimethyl ether, propane, butane, liquid nitrogen, and a combination there of.

19. The cryosurgical instrument of claim 15, wherein the tissue contact end further comprises a guard portion adapted to prevent excessive exposure of the biological tissue surrounding the lesion to the refrigerant.
